(19) United States
(12) Patent Application Publication
(10) Pub. No.: US 2017/0226454 A1
(43) Pub. Date: Aug. 10, 2017

(54) BIOMASS PRODUCTION IN ALKALINE CONDITIONS

(71) Applicant: UTI Limited Partnership, Calgary (CA)

(72) Inventors: Marc STROUS, Calgary (CA); Christine SHARP, Calgary (CA); Hector de la HOZ SIEGLER, Calgary (CA); Gregory WELCH, Calgary (CA)

(73) Assignee: UTI Limited Partnership, Calgary (CA)

(21) Appl. No.: 15/428,800

(22) Filed: Feb. 9, 2017

Related U.S. Application Data
(60) Provisional application No. 62/293,132, filed on Feb. 9, 2016.

(51) Int. Cl. C12M 1/00; C12M 1/107; C12M 1/34

(52) U.S. Cl. CPC C12M 21/02; C12M 23/22; C12M 41/34; C12M 23/36

(57) ABSTRACT

A system and a method for producing biomass from a mixed community of algal species. The method comprises the steps of culturing the mixed community of at least two algal species as biofilms on transparent surfaces having structural features and an optical filter, providing a continuous supply of a culture medium comprising at least 0.5 mol/L aqueous (bi)carbonate and having a pH greater than 9. The method disclosed herein facilitates online monitoring of mixed community productivity by the quantification of oxygen production.

### TECHNICAL FIELD

This disclosure relates to phototrophic production of biomass. More specifically, this disclosure pertains to use of biofilms of phototrophic microbial communities to produce biomass for downstream processing into fuels.

### BACKGROUND

Fossil fuels are a non-renewable fuel source and their combustion results in the emission of the greenhouse gas carbon dioxide, with potential detrimental effects on Earth's ecosystems. Biofuels could offer a sustainable alternative for fossil fuels, yet the growth of terrestrial energy crops has severe environmental and socio-economic consequences. Using aquatic oxygenic microalgae, such as unicellular algae and cyanobacteria, as feedstock for biofuel production eliminates the drawbacks associated with growing terrestrial energy crops. The cultivation of unicellular algae and cyanobacteria does not compete with food or feed crops for arable land and water, since it does not require fertile soil and fresh water. Furthermore, the biomass yield of aquatic oxygenic phototrophs can be about one order of magnitude higher than that of terrestrial crops.

Currently, several aspects of algal production limit a widespread use. For example, the required input of fossil fuels for the construction and operation of algae growth systems often surpasses the energy content of the produced biofuel, resulting in a negative energy balance. The monetary costs of growing algae for biofuel production are also too high to make algal biofuel economically competitive with fossil fuel. Because of its high cost, the current practice of growing algae mainly aims at high value products such as pharmaceuticals and food additives, instead of biofuels.

Large-scale cultivation of photosynthetic microorganisms is usually performed in open ponds, raceway ponds, or tubular photobioreactors. A major drawback of open and raceway ponds is that only low concentrations of cells are achieved. This is caused by limitations in light penetration: only the cells at the very top layer in the pond are exposed to light, while the cells at the lower layers are shaded. This low cell concentration translates in very low volumetric productivities.

Light limitation in tubular photobioreactors is partially alleviated by actively circulating the cells by mixing. Incorporation of mixing leads to increased cell density and reduced light saturation in the cells, but does so at the expense of increased energy input into the cultivation system. Furthermore, oxygen accumulation in the tubular photobioreactors commonly results in the inhibition of photosynthesis.

The poor technological and economic performance of contemporary algal biofuel production systems has been attributed to a number of factors. Operational costs and energy consumption are high because the gas containing the CO₂ needs to be bubbled through bioreactors filled with diluted algae and the operation of the compressors for the gas bubbling consumes electricity. For example, the forced supply of CO₂ can make up ca. 50% of the cost of biomass production in a raceway pond system with a production rate of 3.0-3.6 kg m⁻²d⁻¹ (Slade et al., 2013, Micro-algae cultivation for biofuels: Cost, energy balance, environmental impacts and future prospects. Biomass and Bioenergy: 29-38). Others have estimated the cost of CO₂ inputs to be 36.5% of the total raw materials and utilities cost for the production of dry biomass of Scenedesmus almeriensis at a scale of 200 ton yr⁻¹ (Acién et al., 2012, Production cost of a real microalgae production plant and strategies to reduce it. Biotechnology Advances, 30: 1344-1353).

Downstream processing of suspended algal cells into energy carriers requires an energy- consuming concentration step. Consequently, some processes use algal biomass for anaerobic digestion because less-concentrated algal feedstocks can be used than are required for the extraction of algal lipids for use in biodiesel production. Biogas resulting from anaerobic digestion of algal biomass can be combusted to produce electricity or alternatively, upgraded to obtain the same methane content as natural gas, enabling its use as a transport fuel or its injection into the gas grid. However, upgrading biogas to higher methane content entails significant energy and economic costs.

Algal biotechnology typically also depends on the axenic cultivation of a single strain, such as Spirulina, Nanochloropsis, Chlorella, or Dunaliella. However, at large scale, aseptic conditions are difficult to maintain (Quinn et al., 2012, Nannochloropsis production metrics in a scalable outdoor photobioreactor for commercial applications. Biores. Technol. 117: 164-171) and ecological processes such as invasion by other algae species, decimation by grazers, fungi and/or viral infection lead to process instability (Cauchie et al., 1995, Daphnia magna Straus living in an aerated sewage lagoon as a source of chitin: ecological aspects. Belg. J. Zool. 125; Oswald W J, 1980, Algal production—problems, achievements and potential. Algae biomass: production and use. [sponsored by the National Council for Research and Development, Israel and the Gesellschaft fur Strahlen-und Umweltforschung (GSF), Munich, Germany]; editors, Gedaliah Shelef, Carl J Soeder.), which also decreases economic feasibility.

### SUMMARY

The exemplary embodiments of the present disclosure pertain to methods for growth of biofilms of alkaliphilic microbial communities dominated by phototrophic bacteria for production of biomass for use as feedstocks for fuel production.

According to one aspect, a fuel produced from such biomass feedstocks may be a solid (for example, dried biomass pellets or briquets), a gas (for example, methane) or a liquid (for example, ethanol or biodiesel).

The exemplary methods generally comprise the following elements:
(1) The biofilms are grown on a thin, transparent surface that is exposed to sunlight. CO2 is provided to the biofilms via the alkaline growth medium in the form of sodium and/or potassium (bi)carbonate.
(2) The biofilms comprise diverse microbial communities. To select for a microbial community with favourable properties, the sunlight is attenuated by passing it through an optical filter. The optical filter may be constructed with organic films, preferably with photovoltaic activity. One example of a suitable optical filter is an organic solar cell. By passing sunlight through an optical filter such as an organic solar cell with an organic film having photovoltaic activity, only parts of the solar spectrum are made available to phototrophic microbes. At the same time, the organic photovoltaic activity in the optical filter can use the absorbed photon energy to produce electricity which can be used to operate pumps and other equipment needed to run the overall process, or alternatively, stored in in a battery.
(3) Additional ecologically selective pressure is applied to the biofilms by preventing accumulation of reduced chemical compounds such as sulfide (HS⁻). This may be done by adding nitrate to the growth medium and/or by pumping the growth medium along the biofilms.
(4) The biofilms are harvested periodically by pigging, wiping or by applying a water jet. During harvesting, surface roughness, for example in the form of etched grooves on the transparent surface, ensures that sufficient biomass is left behind for effective regrowth of the biofilms.
(5) The productivity of the biofilms is monitored online by quantification of oxygen production.
(6) The (bi)carbonate in the growth medium is regenerated by capturing CO₂, either from a stack gas or directly from the atmosphere. Regeneration takes place in a separate process module.

### BRIEF DESCRIPTION OF THE FIGURES:

The present disclosure will be described in conjunction with reference to the following drawings in which:

FIG. 1A is a side view and FIG. 1B is a front view of an example of a photobioreactor according to one embodiment of the present invention;

FIG. 2 is a close-up cross-sectional view of the photobioreactor illustrated in FIG. 1;

FIG. 2 is an exemplary illustration of an exemplary process scheme according to one embodiment of the present disclosure;

FIG. 3 is a chart showing the phototrophic microbial productivity in an exemplary bioreactor with an interior depth of 1.6 mm (open circles), 3.5 mm (closed circles) and 7.0 mm (triangles);

FIG. 4A is a pie chart showing distribution of microbial species within a community maintained in a photobioreactor exposed to blues light waves only, while FIG. 4B is a pie chart showing distribution of microbial species within a community maintained in a photobioreactor exposed to white light waves comprising a full solar spectrum, and FIG. 4C is a pie chart showing distribution of microbial species within a community maintained in a photobioreactor exposed to red light waves only;

FIG. 5 is a chart showing the phototrophic microbial productivity in an exemplary bioreactor exposed to red (open triangles) or blue (closed squares) light waves only as compared to white light waves comprising a full solar spectrum (closed circles);

FIG. 6A is a pie chart showing distribution of microbial species within a community maintained in a photobioreactor without regular nutrient media refreshing and regular microbial harvesting, while FIG. 6B is a pie chart showing distribution of microbial species within a community maintained in a photobioreactor with regular nutrient media refreshing and regular microbial harvesting; and

FIG. 7 is a chart showing recovery of phototrophic microbial productivity in an exemplary bioreactor having etched surfaces for supporting microbial growth (open circles) in comparison to phototrophic microbial productivity in an exemplary bioreactor that did not have etched surfaces (closed circles);

### DETAILED DESCRIPTION

The embodiments of the present disclosure generally pertain to integrated systems and processes for the cultivation of biofilms of alkaliphilic microbial communities comprising phototrophic microorganisms for production of biomass for use as feedstocks for fuel production. According to one aspect, a fuel produced from such biomass feedstocks may be a solid (for example, dried biomass pellets) or a gas (for example, methane) or a liquid (for example, ethanol or biodiesel).

Some embodiments pertain to photobioreactors for culturing and maintaining therein said biofilms comprising microbial communities. It is to be noted that the microbial communities will not proliferate within the photobioreactors in the form of suspended cells.

An example of a method according to the present disclosure generally comprise the following steps:
(1) The biofilms are grown on a thin, transparent surface that is exposed to sunlight. CO₂ is provided to the biofilms via the alkaline growth medium in the form of sodium and/or potassium (bi)carbonate.
(2) The biofilms comprise diverse microbial communities. To select a suitable microbial community having favourable properties for biomass production, the sunlight is attenuated by passage through an optical filter. The optical filter may be constructed with organic films, and preferably, may have photovoltaic activity. One example of a suitable optical filter is an organic solar cell. By passing sunlight through an organic solar cell with an organic film having photovoltaic activity, only parts of the solar spectrum are made available to the underlying phototrophic microbes. Examples of suitable optical filters include filters that filter out blue light, or red light, or green light, and other light spectra At the same time, the organic photovoltaic activity in the optical filter can use the absorbed photon energy to produce electricity which can be used to operate pumps and other equipment needed to run the overall process, or alternatively, stored in in a battery.
(3) Additional ecologically selective pressure may be applied to the biofilms by preventing accumulation of reduced chemical compounds such as sulfide (HS⁻). This may be done by adding nitrate to the growth medium and/or by pumping the growth medium along the biofilms.
(4) The biofilms are harvested periodically by pigging, wiping or by applying a water jet to the biofilms. During harvesting, surface roughness, for example in the form of etched grooves on the transparent surface, will ensure that sufficient biomass is left behind for effective regrowth of the biofilms.
(5) The productivity of the biofilms is monitored online by quantification of oxygen production.
(6) The (bi)carbonate in the growth medium is regenerated by capturing CO₂, either from a stack gas or directly from the atmosphere. Regeneration takes place in a separate process module.

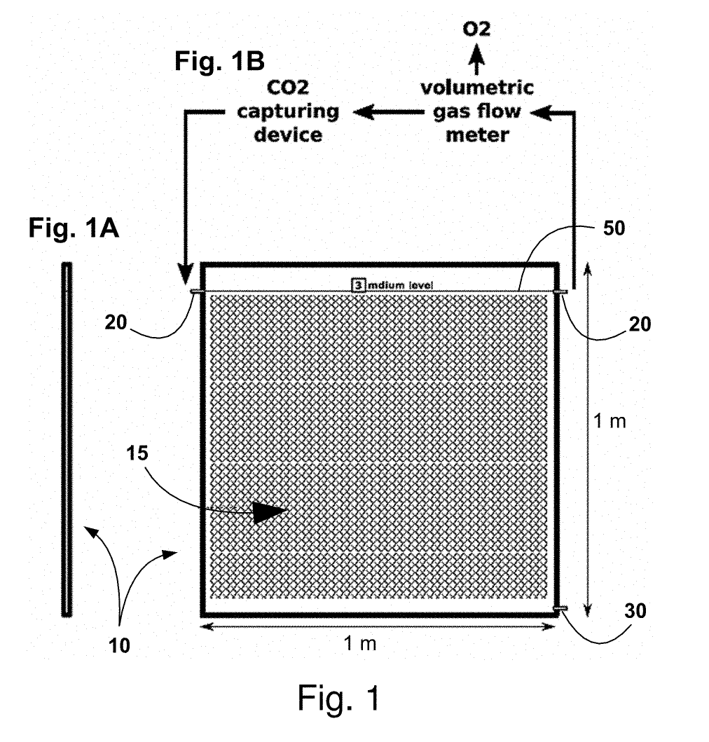

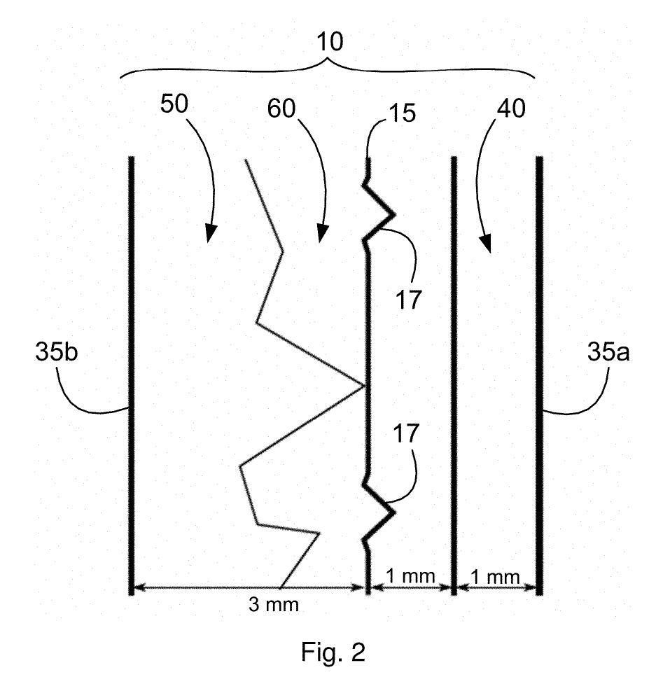

An example of a photobioreactor 10 according to the present disclosure, is shown in FIGS. 1A, 1B, and 2. In this example, the dimensions of the photobioreactor 10 are 1 m high by 1 m wide by 5 mm wide. FIG. 1A shows a side view of the photobioreactor 10 which FIG. 1B shows a front view. The photobioreactor comprises two outer walls 35a, 35b. An organic solar cell 40 about 1-mm thick, is positioned and secured directly adjacent a first outer wall 35a. A transparent sheet material 15 having etched grooves 17 is spaced about 1 mm away from the underside of the organic solar cell 40 and secured in place. The transparent sheet material 15 may be a synthetic polymer such as a polycarbonate resin or alternatively glass or other such sheet materials. This photoreactor 10 has one inlet port 30 receiving therethrough nutrient media and for maintaining nutrient media 50 at a selected level in the photobioreactor 10. One or more outlet ports 20 are provided near the top of the photobioreactor 10 for egress of nutrient media and for periodic harvesting of microbial biomass. The photobioreactor 10 is additionally equipped with piping (not shown) for egress of gases from the top of the photobioreactor 10, with a volumetric gas flow meter, and oxygen egress port, and optionally, a CO₂-capturing device.

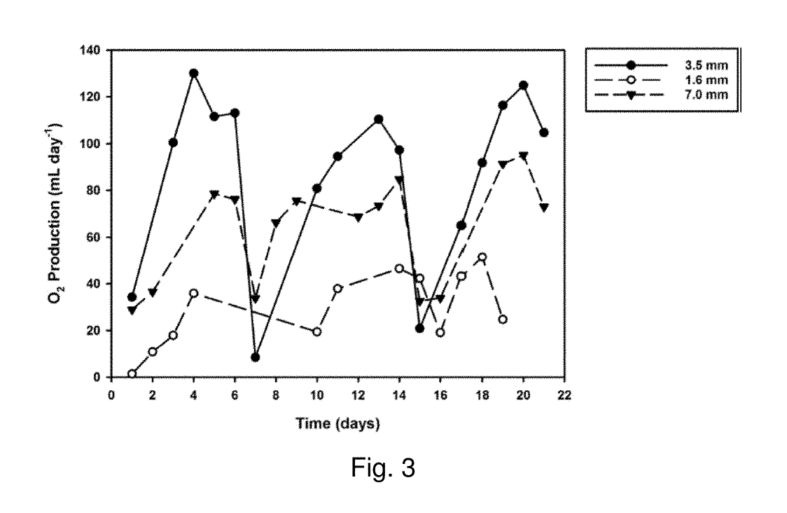

The outer-facing surface of the transparent sheet material 15 (i.e., the face facing the second outer wall 35b) is seeded with a sample of a naturally occurring microbial population collected from a natural habitat, for example, from an alkaline soda lake. Then a selected growth medium containing >0.5 mol/L sodium and/or potassium (bi)carbonate at pH>9 as well as other nutrients suitable to support microbial growth and development. It is to be noted that the photobioreactor modules may be planar and may be mounted vertically or near-vertical, for example at angles of 30° or greater to enable the spontaneous outgassing of the oxygen produced by the biofilms, as oxygen bubbles that collect at the top of the module. A module width of 3.5 mm is ideal to enable the effective outgassing of the produced oxygen (FIG. 3). During outgassing in smaller module widths, gas bubbles may prevent proper affixing of the biofilms to the walls of transparent grooved sheet material and thereby result in lower phototrophic microbial productivity. Larger module widths may also result in lower productivity. However, the dimensions of the photobioreactor may vary in the ranges of 0.5 m to 3.5 m high, 0.5 m to 3.5 m wide, and 2.5 mm to 100 mm wide. It is to be noted that such structures are commonly referred to as “rectangular parellelepipeds”.

As the biofilms develop and produce gases, the gases will flow into the egress piping for separation of 0₂ and CO₂. In a later step, CO₂ may be absorbed from a flue gas or directly from the atmosphere.

Because the CO₂ absorption stage is separate and no gas is provided to the photobioreactor module, the productivity of the photobioreactor can be monitored and quantified online, for example volumetrically as taught by Veiga et al. (1990, A new device for measurement and control of gas production by bench scale anaerobic digesters. Water Res. 24:12, 1551-1554). Oxygen production may be quantified for a single photobioreactor module, or for multiple connected modules. Both the front and back of the module may consist of surfaces as depicted in FIG. 1. Alternatively, only a single side of the module may be transparent. In any case, the transparent surface area of the module is typically arranged such that the sunlight shines onto it at an angle so that ideally, the photosynthetically active radiation remains below 600 μmol/m² surface/s, to limit photoinhibition. That means that the amount of module surface area will be larger than its footprint (typically 2-5×). The modules are also engineered in such a way that rainwater can be collected, stored, and used for the makeup of fresh medium that is lost from the process during biomass harvesting. The module may be seeded with one or more natural microbial communities collected from suitable natural habitats, for example from alkaline soda lakes.

Studies of alkaline soda lakes in Africa and Siberia have shown that both microalgae and cyanobacteria are highly active in such lakes (Seckbach, 2007, Algae and Cyanobacteria in Extreme Environments, Springer; Schragerl et al., 2008, Phytoplankton community relationship to environmental variables in three Kenyan Rift Valley saline-alkaline lakes. Marine and Freshwater Res. 59:125-136) and as such these ecosystems are among the most productive in the world (Melack, 1981, Photosynthetic activity of phytoplankton in tropical African soda lakes. Hydrobiologia 81:71-85.). Alkaline soda lakes typically have moderate to high salt concentrations (sodium carbonate up to saturation) and pHs ranging from 9 to 11 with diverse microbial communities (Sorokin et al., 2014, Microbial diversity and biogeochemical cycling in soda lakes. Extremophiles 18:791-809.). Many small alkaline lakes harbouring active photosynthetic microbial mats adapted to high pH and alkalinity have been discovered on the Cariboo Plateau in British Columbia, Canada (Brady et al., 2013, Isotopic biosignatures in carbonate-rich, cyanobacteria dominated mats of the Cariboo Plateau, B.C. Geobiology 11:437-456). The pHs of the Cariboo lakes show little variation seasonally and over successive years with mean values ranging between 10.1 to 10.2±0.1. The lakes are dominated by Na⁺ ions with Na⁺ concentrations ranging from 6,508 to 32,600 mg L⁻¹ and are poor in Ca²⁺ ions (<10 mg L⁻¹) and Mg²⁺ ions (<96 mg L⁻¹). The maximum dissolved inorganic carbon concentration observed is 9,200 mg L⁻¹.

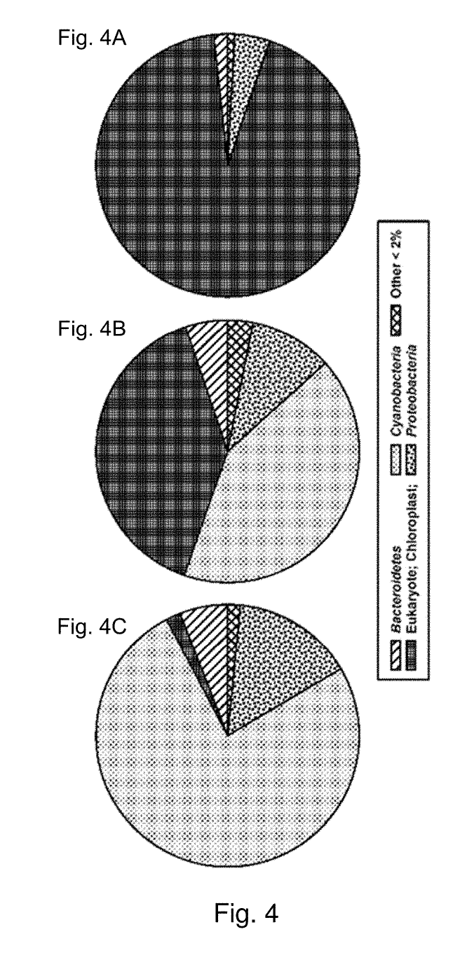

Multiple different types of microbial phototrophs may be active at the same time in a microbial community, and may compete for space, nutrients, and light. These different types of phototrophs typically have different properties with respect to their density, biofilm structural strength, productivity, and lipid content. Different types of phototrophs use different parts of the solar spectrum. For example, cyanobacteria mainly use red light whereas diatoms also use blue light. These different phototrophs may also interact in antagonistic ways, resulting in a loss of productivity. To select for a specific type of desired phototroph, the sunlight is attenuated by the addition of an optic filter on the outside transparent wall of a photobioreactor module as shown in FIGS. 4A, 4C. This filter preferentially transmits light either above or below 625 nm.

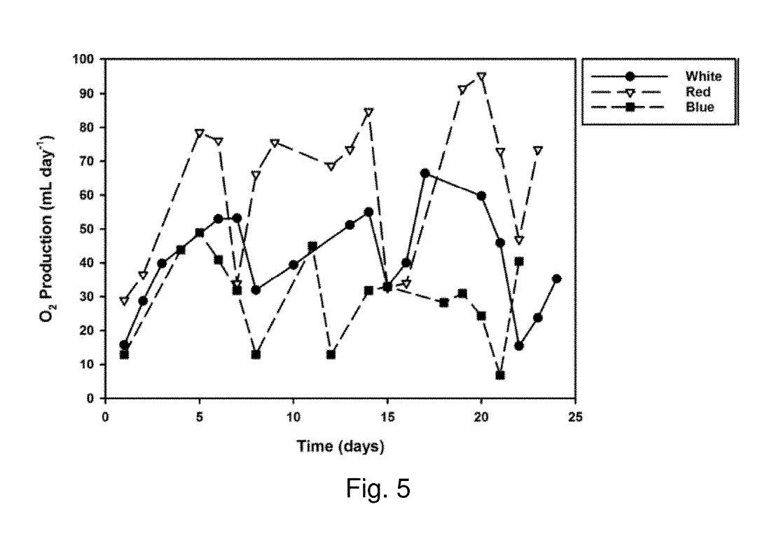

The most productive microbial community was grown under red light (>625 nm, as opposed to white or blue; FIG. 5) and was dominated by oxygenic phototrophs closely related to Lyngbya sp.

This microbial community also contained members of the genera Marinicella and Rhodobaca (phylum Proteobacteria), the family Saprospiraceae (phylum Bacteroidetes) and order Oceanospirillales (phylum Proteobacteria) (Table 1). The addition of an optic filter transmitting light below 625 nm selects for a phototrophic microbial community dominated by diatoms (FIG. 4A) that is less productive than either the red light wave community or the full spectrum white light wave community (FIG. 5). The less productive blue light microbial community was dominated by the Eukaryote genus Nitzschia (phylum Bacillariophyta) with lower amounts of the bacterial genera Marinicella, Alcanivorax, and Rhodobaca (phylum Proteobacteria) and family Saprospiraceae (phylum Bacteroidetes) (Table 1). The full spectrum white light microbial community was a mixture of the microbial communities found in the blue and red light microbial communities (Table 1).

Preferentially, the filter consists of a semi-transparent photovoltaic device that converts the energy in the solar spectrum not used by the desired microbial phototrophs into electricity. Organic and dye-based photovoltaics are energy conversion technologies that rely on organic materials to convert sunlight into electricity. These organic materials are highly soluble in organic solvents which allows for room temperature solution deposition, thus enabling the fabrication of solar cell devices onto a range of substrates/surfaces including foils and plastic films that are light weight and flexible. In addition these cells can be color-tuned and made semi-transparent. Thus, the type and amount of transmitted light can be finely adjusted. The electricity so produced may be used to power pumps and other equipment needed to run the photobioreactor. Thus, the filter differs from ones previously disclosed that reflect light (for example, such as those disclosed in US Pub. Pat. Appl. No. 2014/0154769 A1).

TABLE 1
Summary of the composition of microbial communities grown in biofilms under
red, white, or blue wavelength light.

| Red | White | Blue | Kingdom | Phylum | Class | Order |
|---|---|---|---|---|---|---|
| 0.28 | 0.57 | 0.02 | Bacteria | | | |
| 0.00 | 0.06 | 0.02 | Bacteria | BD1-5 | | |
| 0.22 | 0.17 | 0.10 | Bacteria | Bacteroidetes | | |
| 0.06 | 0.00 | 0.00 | Bacteria | Bacteroidetes | Bacteroidia | Bacteroidales |
| 0.00 | 0.00 | 0.02 | Bacteria | Bacteroidetes | Bacteroidia | Bacteroidales |
| 0.23 | 0.11 | 0.00 | Bacteria | Bacteroidetes | Cytophagia | |
| 0.06 | 0.00 | 0.00 | Bacteria | Bacteroidetes | Cytophagia | Cytophagales |
| 0.05 | 0.06 | 0.02 | Bacteria | Bacteroidetes | Cytophagia | Order_III |
| 0.05 | 0.17 | 0.02 | Bacteria | Bacteroidetes | Cytophagia | Order_III |
| 0.67 | 1.20 | 0.25 | Bacteria | Bacteroidetes | Cytophagia | Order_III |
| 0.28 | 0.29 | 0.00 | Bacteria | Bacteroidetes | Flavobacteriia | Flavobacteriales |
| 0.45 | 0.06 | 0.41 | Bacteria | Bacteroidetes | Flavobacteriia | Flavobacteriales |
| 0.67 | 0.34 | 0.00 | Bacteria | Bacteroidetes | Flavobacteriia | Flavobacteriales |
| 0.13 | 0.46 | 0.08 | Bacteria | Bacteroidetes | Flavobacteriia | Flavobacteriales |
| 3.05 | 2.35 | 0.83 | Bacteria | Bacteroidetes | Sphingobacteriia | Sphingobacteriales |
| 0.02 | 0.00 | 0.00 | Bacteria | Candidate_division_OD1 | | |
| 0.00 | 0.00 | 0.02 | Bacteria | Candidate_division_SR1 | | |
| 0.02 | 0.00 | 0.00 | Bacteria | Candidate_division_WS6 | | |
| 0.13 | 0.00 | 0.04 | Bacteria | Chlamydiae | Chlamydiae | Chlamydiales |
| 0.03 | 0.00 | 0.00 | Bacteria | Chlamydiae | Chlamydiae | Chlamydiales |
| 0.03 | 0.00 | 0.00 | Bacteria | Chloroflexi | Anaerolineae | Anaerolineales |
| 0.00 | 0.06 | 0.00 | Bacteria | Chloroflexi | Thermomicrobia | Sphaerobacterales |
| 1.80 | 39.41 | 93.09 | Eukaryote | Bacillariophyta | Bacillariophyceae | Bacillariales |
| 0.75 | 0.86 | 0.00 | Bacteria | Cyanobacteria | Cyanobacteria | |
| 0.52 | 2.01 | 0.00 | Bacteria | Cyanobacteria | Cyanobacteria | SubsectionI |
| 0.06 | 0.00 | 0.00 | Bacteria | Cyanobacteria | Cyanobacteria | SubsectionIII |
| 74.43 | 39.19 | 0.04 | Bacteria | Cyanobacteria | Cyanobacteria | SubsectionIII |
| 0.20 | 0.11 | 0.00 | Bacteria | Deinococcus-Thermus | Deinococci | Deinococcales |
| 0.00 | 0.06 | 0.00 | Bacteria | Firmicutes | Clostridia | Clostridiales |
| 0.06 | 0.06 | 0.08 | Bacteria | Gemmatimonadetes | Gemmatimonadetes | BD2-11_terrestrial_group |
| 0.05 | 0.00 | 0.02 | Bacteria | Lentisphaerae | SS1-B-03-39 | |
| 0.14 | 0.00 | 0.10 | Bacteria | Planctomycetes | Phycisphaerae | Phycisphaerales |
| 0.17 | 0.63 | 0.27 | Bacteria | Planctomycetes | Phycisphaerae | Phycisphaerales |
| 0.08 | 0.00 | 0.08 | Bacteria | Proteobacteria | | |
| 0.00 | 0.11 | 0.00 | Bacteria | Proteobacteria | Alphaproteobacteria | |
| 0.05 | 0.06 | 0.00 | Bacteria | Proteobacteria | Alphaproteobacteria | Caulobacterales |
| 0.06 | 0.17 | 0.02 | Bacteria | Proteobacteria | Alphaproteobacteria | Caulobacterales |
| 0.33 | 0.17 | 0.06 | Bacteria | Proteobacteria | Alphaproteobacteria | DB1-14 |
| 1.45 | 0.98 | 0.42 | Bacteria | Proteobacteria | Alphaproteobacteria | Rhizobiales |
| 0.03 | 0.06 | 0.06 | Bacteria | Proteobacteria | Alphaproteobacteria | Rhizobiales |
| 0.23 | 0.17 | 0.04 | Bacteria | Proteobacteria | Alphaproteobacteria | Rhodobacterales |
| 2.31 | 2.18 | 0.50 | Bacteria | Proteobacteria | Alphaproteobacteria | Rhodobacterales |
| 0.34 | 0.69 | 0.08 | Bacteria | Proteobacteria | Alphaproteobacteria | Rhodobacterales |
| 0.11 | 0.23 | 0.02 | Bacteria | Proteobacteria | Alphaproteobacteria | Rickettsiales |
| 0.03 | 0.00 | 0.06 | Bacteria | Proteobacteria | Deltaproteobacteria | Bdellovibrionales |
| 0.09 | 0.00 | 0.12 | Bacteria | Proteobacteria | Deltaproteobacteria | Bdellovibrionales |
| 0.02 | 0.00 | 0.00 | Bacteria | Proteobacteria | Deltaproteobacteria | Bdellovibrionales |
| 0.02 | 0.11 | 0.00 | Bacteria | Proteobacteria | Deltaproteobacteria | Desulfuromonadales |
| 0.73 | 0.40 | 0.12 | Bacteria | Proteobacteria | Gammaproteobacteria | |
| 0.97 | 0.11 | 0.15 | Bacteria | Proteobacteria | Gammaproteobacteria | Alteromonadales |
| 0.00 | 0.00 | 0.04 | Bacteria | Proteobacteria | Gammaproteobacteria | Alteromonadales |
| 0.25 | 0.17 | 0.00 | Bacteria | Proteobacteria | Gammaproteobacteria | Alteromonadales |
| 0.02 | 0.00 | 0.00 | Bacteria | Proteobacteria | Gammaproteobacteria | Chromatiales |
| 0.06 | 0.00 | 0.02 | Bacteria | Proteobacteria | Gammaproteobacteria | Incertae_Sedis |
| 0.77 | 0.34 | 0.35 | Bacteria | Proteobacteria | Gammaproteobacteria | Incertae_Sedis |
| 0.02 | 0.00 | 0.02 | Bacteria | Proteobacteria | Gammaproteobacteria | HOC36 |
| 0.03 | 0.00 | 0.04 | Bacteria | Proteobacteria | Gammaproteobacteria | Oceanospirillales |
| 0.31 | 0.17 | 0.56 | Bacteria | Proteobacteria | Gammaproteobacteria | Oceanospirillales |
| 0.02 | 0.06 | 0.04 | Bacteria | Proteobacteria | Gammaproteobacteria | Oceanospirillales |
| 2.09 | 0.52 | 0.04 | Bacteria | Proteobacteria | Gammaproteobacteria | Oceanospirillales |
| 0.42 | 0.34 | 0.06 | Bacteria | Proteobacteria | Gammaproteobacteria | Oceanospirillales |
| 0.05 | 0.00 | 0.00 | Bacteria | Proteobacteria | Gammaproteobacteria | Oceanospirillales |
| 0.00 | 0.00 | 0.06 | Bacteria | Proteobacteria | Gammaproteobacteria | Oceanospirillales |
| 0.03 | 0.00 | 0.00 | Bacteria | Proteobacteria | Gammaproteobacteria | Oceanospirillales |
| 4.02 | 3.16 | 1.27 | Bacteria | Proteobacteria | | |
| 0.03 | 0.00 | 0.10 | Bacteria | Proteobacteria | Gammaproteobacteria | Pseudomonadales |
| 0.03 | 0.00 | 0.00 | Bacteria | Spirochaetae | Spirochaetes | Spirochaetales |
| 0.16 | 0.00 | 0.00 | Bacteria | Spirochaetae | Spirochaetes | Spirochaetales |
| 0.03 | 0.11 | 0.00 | Bacteria | Verrucomicrobia | Opitutae | |
| 0.17 | 0.06 | 0.00 | Bacteria | Verrucomicrobia | Opitutae | BC-COM435 |
| 0.03 | 1.09 | 0.19 | Bacteria | Verrucomicrobia | Opitutae | Puniceicoccales |
| 0.00 | 0.06 | 0.04 | Bacteria | Verrucomicrobia | Opitutae | Puniceicoccales |
| 0.00 | 0.06 | 0.00 | Bacteria | Verrucomicrobia | Opitutae | Puniceicoccales |
| 0.00 | 0.17 | 0.06 | Bacteria | Verrucomicrobia | Verrucomicrobiae | Verrucomicrobiales |

| Red | White | Blue | Family | Genus |
|---|---|---|---|---|
| 0.28 | 0.57 | 0.02 | | |
| 0.00 | 0.06 | 0.02 | | |
| 0.22 | 0.17 | 0.10 | | |
| 0.06 | 0.00 | 0.00 | | |
| 0.00 | 0.00 | 0.02 | ML635J-40 | |
| 0.23 | 0.11 | 0.00 | | |
| 0.06 | 0.00 | 0.00 | | |
| 0.05 | 0.06 | 0.02 | | |
| 0.05 | 0.17 | 0.02 | F1-37X2 | |
| 0.67 | 1.20 | 0.25 | ML310M-34 | |
| 0.28 | 0.29 | 0.00 | Cryomorphaceae | Brumimicrobium |
| 0.45 | 0.06 | 0.41 | Cryomorphaceae | Fluviicola |
| 0.67 | 0.34 | 0.00 | Cryomorphaceae | Owenweeksia |
| 0.13 | 0.46 | 0.08 | Flavobacteriaceae | Psychroflexus |
| 3.05 | 2.35 | 0.83 | Saprospiraceae | |
| 0.02 | 0.00 | 0.00 | | |
| 0.00 | 0.00 | 0.02 | | |
| 0.02 | 0.00 | 0.00 | | |
| 0.13 | 0.00 | 0.04 | | |
| 0.03 | 0.00 | 0.00 | Waddliaceae | Waddlia |
| 0.03 | 0.00 | 0.00 | Anaerolineaceae | |
| 0.00 | 0.06 | 0.00 | Sphaerobacteraceae | Nitrolancea |
| 1.80 | 39.41 | 93.09 | Bacillariaceae | Nitzschia |
| 0.75 | 0.86 | 0.00 | | |
| 0.52 | 2.01 | 0.00 | FamilyI | Cyanobacterium |
| 0.06 | 0.00 | 0.00 | FamilyI | |
| 74.43 | 39.19 | 0.04 | FamilyI | Lyngbya |
| 0.20 | 0.11 | 0.00 | Trueperaceae | Truepera |
| 0.00 | 0.06 | 0.00 | | |
| 0.06 | 0.06 | 0.08 | | |
| 0.05 | 0.00 | 0.02 | | |
| 0.14 | 0.00 | 0.10 | Phycisphaeraceae | SM1A02 |
| 0.17 | 0.63 | 0.27 | Phycisphaeraceae | Urania-1B-19 |
| 0.08 | 0.00 | 0.08 | | |
| 0.00 | 0.11 | 0.00 | | |
| 0.05 | 0.06 | 0.00 | Hyphomonadaceae | Glycocaulis |
| 0.06 | 0.17 | 0.02 | Hyphomonadaceae | Oceanicaulis |
| 0.33 | 0.17 | 0.06 | | |
| 1.45 | 0.98 | 0.42 | Bradyrhizobiaceae | Salinarimonas |
| 0.03 | 0.06 | 0.06 | Phyllobacteriaceae | Pseudaminobacter |
| 0.23 | 0.17 | 0.04 | Rhodobacteraceae | |
| 2.31 | 2.18 | 0.50 | Rhodobacteraceae | Rhodobaca |
| 0.34 | 0.69 | 0.08 | Rhodobacteraceae | Rhodovulum |
| 0.11 | 0.23 | 0.02 | | |
| 0.03 | 0.00 | 0.06 | Bacteriovoracaceae | Bacteriovorax |
| 0.09 | 0.00 | 0.12 | Bacteriovoracaceae | Peredibacter |
| 0.02 | 0.00 | 0.00 | Bdellovibrionaceae | Bdellovibrio |
| 0.02 | 0.11 | 0.00 | GR-WP33-58 | |
| 0.73 | 0.40 | 0.12 | | |
| 0.97 | 0.11 | 0.15 | Alteromonadaceae | Marinobacter |
| 0.00 | 0.00 | 0.04 | Alteromonadaceae | Simiduia |
| 0.25 | 0.17 | 0.00 | Idiomarinaceae | Aliidiomarina |
| 0.02 | 0.00 | 0.00 | Ectothiorhodospiraceae | Ectothiorhodospira |
| 0.06 | 0.00 | 0.02 | | Alkalimonas |
| 0.77 | 0.34 | 0.35 | | Methylonatrum |
| 0.02 | 0.00 | 0.02 | | |
| 0.03 | 0.00 | 0.04 | | |
| 0.31 | 0.17 | 0.56 | Alcanivoracaceae | Alcanivorax |
| 0.02 | 0.06 | 0.04 | Halomonadaceae | Halomonas |
| 2.09 | 0.52 | 0.04 | ML617.5J-3 | |
| 0.42 | 0.34 | 0.06 | OM182_clade | |
| 0.05 | 0.00 | 0.00 | Oceanospirillaceae | Marinospirillum |
| 0.00 | 0.00 | 0.06 | Oceanospirillaceae | Nitrincola |
| 0.03 | 0.00 | 0.00 | Oceanospirillaceae | Pseudospirillum |
| 4.02 | 3.16 | 1.27 | Incertae_Sedis | Marinicella |
| 0.03 | 0.00 | 0.10 | Pseudomonadaceae | Pseudomonas |
| 0.03 | 0.00 | 0.00 | PL-11B10 | |
| 0.16 | 0.00 | 0.00 | Spirochaetaceae | Spirochaeta |
| 0.03 | 0.11 | 0.00 | | |
| 0.17 | 0.06 | 0.00 | | |
| 0.03 | 1.09 | 0.19 | Puniceicoccaceae | |
| 0.00 | 0.06 | 0.04 | Puniceicoccaceae | Coraliomargarita |
| 0.00 | 0.06 | 0.00 | Puniceicoccaceae | marine_group |
| 0.00 | 0.17 | 0.06 | Verrucomicrobiaceae | Haloferula |

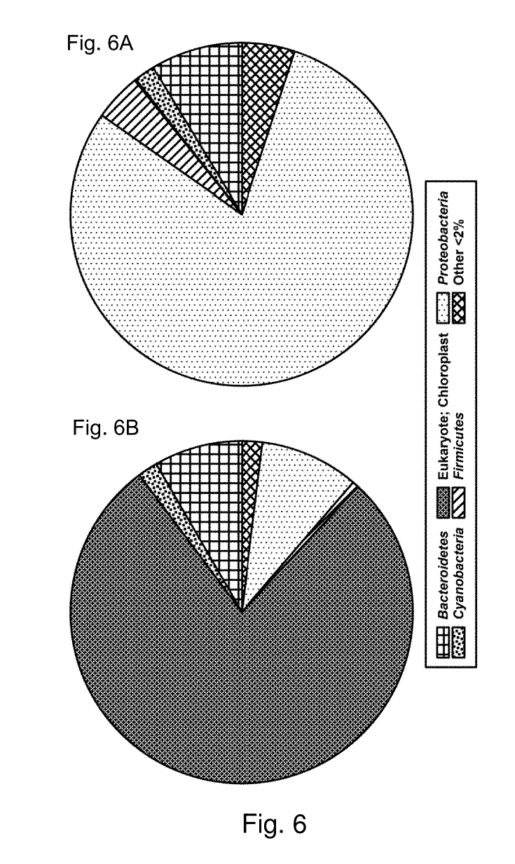

Microbial communities growing in biofilms contain oxygenic phototrophic microbes that contribute to productivity. However, biofilms also contain other microbes that might negatively affect productivity. For example, sulfate-reducing bacteria may be present. When parts of the biofilms become anoxic (for example, at night when no oxygen is produced), sulfate-reducing bacteria produce sulfide which is toxic to the oxygenic phototrophic microbes. To limit toxic effects of anoxic conditions inside biofilms, the oxic medium is pumped along the biofilms with a pump (typically 1 to 4 volume changes/day). Flow can be applied continuously or periodically and has the additional benefits of: (i) cooling the biofilms when their temperature becomes too high because of the exposure to sunlight during the day, and (ii) removal of excess oxygen thereby preventing inhibition of the biofilms' biological activities. In the absence of sufficient flow, a microbial community with low productivity is selected (FIGS. 6A, 6B). In addition, a dissolved redox buffer (e.g. nitrate or iron(II/III), 5-25 mM) is added to the medium to select for anaerobic bacteria that may out compete sulfate reducers for substrates and/or re-oxidize any sulfide that is still produced.

Additional measures to select for biomass with favourable properties or to reduce process costs can be implemented. For example, a nitrogen source may be omitted from the growth medium to stimulate nitrogen fixation.

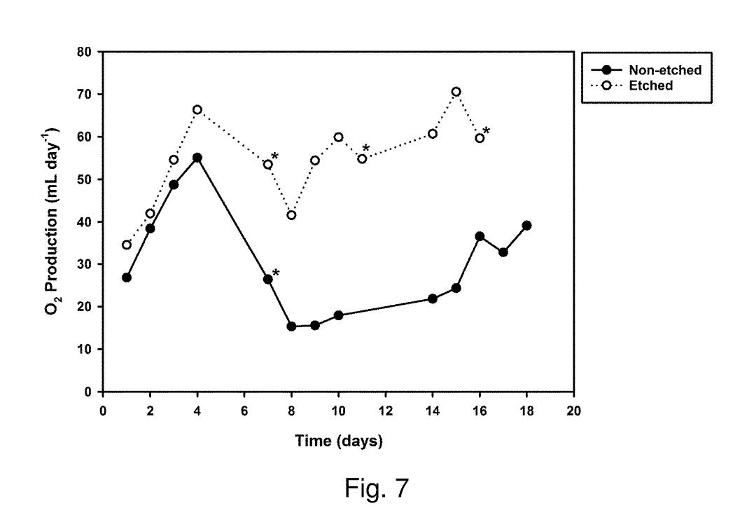

The biofilms are harvested periodically, for example by applying mechanical or hydraulic force, for example by “pigging” (as is done for pipelines), mechanical wiping, or by application of hydraulic shear with a water jet. To enable effective and rapid re-growth of the biofilms after harvesting, macroscopic structural features (>50 μm in size) are present on the transparent walls of the photobioreactor. These features are macroscopic. In one embodiment of the method disclosed herein, the structural features are present in the form of grooves (17 in FIG. 2). Because of these features, some of the microorganisms remain attached to the surface during harvesting and act as seed for reestablishment and development of a new biofilm (FIG. 7).

The harvested microbial biomass may be dried and then compressed to form combustible additionally comprising the steps of drying the harvested microbial biomass, and compressing the dried microbial biomass to form a plurality of combustible pellets or briquets or other such materials. Alternatively, the harvested biomass may be extruded through suitable dies to form elongate strands to form pellets that may then dried to a combustible material form.

Alternatively, the harvested microbial biomass may be used as a feedstock for a fermentation process for production of a fuel ethanol therefrom. Alternatively, the harvested microbial biomass may be anaerobically digested to produce one or more combustible gases therefrom.

1. A method for producing microbial biomass for use as a fuel feedstock, comprising the steps of:
providing a photobioreactor having an optical filter adjacent to and extending along the inner face of an outer wall of the photobioreactor, and a transparent sheet material adjacent to and extending along the optical filter surface, wherein the transparent sheet material has macroscopic structural features formed thereinto;
providing a circulating supply of a culture medium through the photobioreactor, the culture medium comprising: (i) nutrients for supporting microbial growth and/or metabolism, (ii) at least 0.5 mol/L aqueous (bi)carbonate, (iii) a redox buffer in the form of a nitrate or a dissolved iron, and (iv) having a pH greater than 9;
culturing at least two microbial species in the circulating supply of culture medium on the transparent sheet material to form a biofilm thereon;
selectively harvesting microbial biomass from the photobioreactor.

2. The method according to claim 1, wherein the photobioreactor has a rectangular parellelepiped shape.

3. The method according to claim 1, additionally comprising a step of quantifying oxygen produced by the biofilm as a measure of online productivity;

4. The method according to claim 1, wherein the macroscopic structural features are in the form of grooves.

5. The method according to claim 1, wherein the optical filter comprises a transparent organic photovoltaic device.

6. The method according to claim 1, wherein the optical filter filters out blue light or red light.

7. The method according to claim 1, wherein the circulating supply of a culture medium comprises a redox buffer.

8. The method according to claim 1, wherein the photobioreactor is arranged at an angle of at least 30° relative to a horizontal axis.

9. The method according to claim 1, wherein the at least two microbial species includes at least one microbial species from bacterial genus Lyngbya of at least one species from eukaryote genus Nitzschia.

10. The method according to claim 1, additionally comprising the steps of drying the harvested microbial biomass, and compressing the dried microbial biomass to form a combustible material therefrom.

11. The method according to claim 1, additionally comprising the steps of providing the harvested microbial biomass as a feedstock for a fermentation process for production of a fuel ethanol therefrom.

12. The method according to claim 1, additionally comprising the steps of anaerobically digesting the harvested microbial biomass to produce one or more combustible gases therefrom.

13. A photobioreactor for production of microbial biomass, comprising:
a chamber having opposing outer walls, a base portion, and a top portion;
an optical filter adjacent extending along an inner face of one of the opposing outer walls;
a transparent sheet material adjacent to and extending along the optical filter surface, wherein the transparent sheet material has macroscopic structural features formed thereinto;
at least one inlet port approximate the top portion of the photobioreactor;
an outlet port approximate the pase portion of the photoreactor; and
a piping in communication with the photobioreactor for receiving gases therefrom.

14. A photobioreactor according to claim 13, wherein the chamber forms a rectangular parellelepiped shape.

15. A photobioreactor according to claim 13, wherein the piping is in communication with an oxygen meter.

16. A photobioreactor according to claim 13, wherein the piping is in communication with a CO₂-capturing device.

17. A photobioreactor according to claim 13, wherein the optical filter comprises a photovoltaic cell.

18. A photobioreactor according to claim 13, wherein the optical filter comprises an organic solar cell.

19. A photobioreactor according to claim 13, wherein the optical filter filters out blue light or red light.
